(19) United States
(12) Patent Application Publication
(10) Pub. No.: US 2005/0288555 A1
(43) Pub. Date: Dec. 29, 2005

(54) Methods and devices for illuminating, vievwing and monitoring a body cavity

(76) Inventor: Kenneth E. Binmoeller, Rancho Sante Fe, CA (US)

Correspondence Address: SCOTT EVANS, 1252 COUNTRY HILLS DR., SANTA ANA, CA, 92705, US

(21) Appl. No.: 11/167,699

(22) Filed: Jun. 27, 2005

Related U.S. Application Data
(60) Provisional application No. 60/584,325, filed on Jun. 28, 2004.

(51) Int. Cl. A61B 1/06; A61B 5/00

(52) U.S. Cl. 600/160; 600/109; 600/300

(57) ABSTRACT

A device and method for illuminating, viewing and monitoring internal body surfaces without an external attachment is disclosed. The present device includes a housing that is suitable for swallowing by the patient or by placement with an endoscope, either through the working lumen or attached to the endoscope. The device has an optical or sensor element coupled to the housing that can be used for illuminating, visualizing or monitoring a body surface. A power source may be located in the housing to provide power to the optical or sensor element. The device may be secured to the body using securement elements.

### PRIORITY INFORMATION

This application claims priority to U.S. Provisional Application No. 60/584,325 filed on Jun. 28, 2004, the entire contents of which are hereby expressly incorporated by reference.

### BACKGROUND OF THE INVENTION

1. Field of the Invention

The present invention relates to self contained devices for illuminating, viewing and monitoring hollow cavities of the body. More particularly the present invention relates to apparatuses and methods for performing these functions without the need for an external connection to the device.

2. Description of the Related Art

It is often necessary in various endoscopic or laparoscopic procedures, to view an internal body cavity to examine the internal surfaces of the hollow cavity and to assess the need for further intervention. When further intervention is required, the surgical site requires illumination and a method for the physician to view the site. The typical laparoscopic or flexible endoscope employed in these procedures has several components; an illumination system, a viewing element that transmits an image from the distal end of the scope to the proximal end, and a working lumen for passing surgical instruments into the body. The illumination system in most endoscopes, for example, is comprised of an external light source, commonly a xenon or halogen lamp. The endoscope is then connected with the external light source at its proximal end and the light is transported to the distal end of the endoscope using a fiber optic bundle which transmits the light from a proximal position outside the patient's body to a distal end of the endoscope in order to illuminate the observation space with light. For bright illumination, white light is usually used, but for other applications, e.g. examinations with excitation light, colored light is also used. The viewing element in most endoscopes consists of one of two types; an optical imaging system consisting of a lens, fiber optic bundle and an eyepiece or an electronic imaging system that uses an electronic chip (CCD) at the tip of the endoscope to deliver images electronically through wires to a video monitor. The working lumen should be as large as possible to permit the introduction of diagnostic and therapeutic instruments such as biopsy forceps, snares, loops, clips, and dilation balloons into the interior cavities of the body.

Present day endoscopes have a significant limitation in that the diameter of the working lumen restricts the number and size of the instruments that can be safely placed through it. However, endoscopic procedures are becoming more common and many of these procedures are using instruments with ever increasing diameters. Since the illumination system and the viewing element occupy significant portions of the cross sectional area of an endoscope, there is limited area available for developing larger working lumens.

In light of the foregoing, there is a need for a better designed endoscope having an enlarged working lumen. If the illumination element and/or the viewing element could be removed from the endoscope, significant space would be freed up for a larger working lumen and a significant barrier to the advancement of endoscopic interventional procedures could be removed.

### BRIEF SUMMARY OF THE INVENTION

Accordingly the present invention is directed to devices and methods that reduce the need for placing the illumination element and/or viewing element in an endoscope as described above. The device and method described herein replace these elements and permit the placement of the necessary illumination and/or viewing elements at a remote location in the body cavity. This reduces the need for a dedicated light source, fiber optics and/or electronic imaging sensors inside the endoscope. This means that the endoscope can be constructed with a larger working lumen to permit larger or a greater number of working instruments to be passed through the lumen and into the body cavity by the physician.

The present invention is directed to a device that, as embodied and broadly described herein, includes a housing that is suitable for placement with the assistance of an endoscope or a laparoscope, either through the working lumen or attached to the tip, or by the patient swallowing the device directly. The device may have an optical or sensor element coupled to the housing and a power source located in the housing to provide power to the optical or sensor element.

The optical element can include at least one light source and/or a viewing element for illuminating and viewing a body cavity. The light source may have a lens that can be used to magnify or diffuse light from the light source. The housing may also have a securement element that is useful for securing the device to the walls of the body cavity.

The sensor element can be a device to monitor various chemical and physical properties of the cavity. Examples of important parameters that might be useful to monitor include: pH, force of contractility, temperature, enzyme, metabolite or protein concentrations, etc.

In another aspect of the invention, the optical or sensor element may articulate away from the housing at multiple points so that multi axis views or sensory input can be obtained from the body cavity. If both a light source and a viewing element are incorporated into the optical element, it is possible that the light source and the viewing element might be articulated so that these elements are on different axes.

In another aspect the present invention includes a method of viewing a body cavity of a patient. In the method the device, which includes a housing, is positioned in a body cavity and the housing is secured to the wall of the body cavity. A light source coupled to the housing illuminates the body cavity and a viewing element transmits images of the body cavity to a receiver positioned outside the patient's body.

In another aspect the present invention includes a method of measuring a condition of a body cavity of a patient. In the method the device, which includes a housing, is positioned in a body cavity and the housing is secured to the wall of the body cavity. A sensor coupled to the housing measures the condition of the body cavity and transmits this information to a receiver positioned outside the patient's body.

It is to be understood that both the foregoing general description and the following detailed description are exemplary and are intended to provide further explanation of the invention as claimed.

### BRIEF DESCRIPTION OF THE DRAWINGS

The accompanying drawings are included to provide a further understanding of the invention and are incorporated in and constitute a part of this specification. The drawings illustrate embodiments of the invention and, together with the description, serve to explain the principles of the invention. In the drawings,

FIG. 1 is a perspective view of one embodiment of an apparatus for illuminating a body cavity.

FIG. 2a is a detail view of the optical element assembly having a lens.

FIG. 2b is a detail view of an alternate light source without a lens.

FIG. 2c is a detail view of a multiple light source configuration without a lens.

FIG. 3 is a section view of the housing showing the power source and switch.

FIG. 4 is a section view of an optical element having a viewing element and transmitter.

FIG. 5 is a detail view of one embodiment of a securement element utilizing loops.

FIG. 6a is a detail view of another embodiment of a securement element utilizing a hook shown in a closed position.

FIG. 6b is a detail view of the securement element of FIG. 6a with the hook shown in a partially open position.

FIG. 6c is a detail view of the securement element of FIG. 6a shown with two hooks in a grasping position.

FIG. 7a is a perspective view of another embodiment of the securement element shown in the open position.

FIG. 7b is a detail view of the securement element of FIG. 7a shown with integral pincers.

FIG. 8 is a perspective view of another embodiment of a securement element showing an endoscope with a magnetic end portion.

FIG. 9a is a detail view of another embodiment of the securement element shown in the closed position.

FIG. 9b is a detail view of the securement element of FIG. 9a shown fully extended inside a body vessel.

FIG. 10 is a section view of the device tethered to a vessel wall using an alternate method.

FIG. 11a is a view of another embodiment of the present invention shown in the closed position.

FIG. 11b is a view of the embodiment of FIG. 11a shown in the open position.

FIG. 12 is a view of another embodiment of a securement element with a helicoil or screw.

FIG. 13 is a view of another embodiment of a securement element with legs to align the sensor or optical element.

FIG. 14 is a view of a delivery system to deliver the apparatus shown in FIG. 13.

FIG. 15 is a view of another embodiment of a securement element with an inflatable orientation portion.

FIG. 16 is a view of another embodiment of the present invention with a coil shaped illumination tube.

FIG. 17 is a view of another embodiment of the present invention with a weighted segment in the sensor housing.

FIG. 18 is a view of another embodiment of the present invention with a suction chamber and securement element.

FIG. 19 is a view of another embodiment of the present invention with a magnetic implant securement element.

### DETAILED DESCRIPTION OF THE INVENTION

Reference will now be made in detail to the present preferred embodiments of the invention, examples of which are illustrated in the accompanying drawings. Wherever possible the same reference numbers are used in the drawings and the description to refer to the same or like parts, and similar reference numerals are used to refer to similar elements.

The devices and methods described herein may offer improvements over the techniques currently utilized to perform endoscopy procedures. In one embodiment the apparatus is a self contained illumination device comprised of a housing, a light source coupled to the housing, a battery located in the housing and a securement element for attaching the housing to tissue. In another embodiment the apparatus is a self contained optical imaging unit, comprised of a housing, an optical element including a viewing element and a transmitter, a battery located in the housing, and a securing element for attaching the housing to tissue. In another embodiment the previous two embodiments are combined into an apparatus comprised of a housing, light source coupled to the housing, an optical element including a viewing element, a transmitter, a battery located in the housing and a securing element for attaching the housing to tissue. In yet another embodiment the apparatus is comprised of a housing, a sensor for monitoring chemical and/or physical properties of the cavity and its contents, a transmitter, a battery located in the housing and a securing element for attaching the housing to tissue.

All these devices are intended to be small enough to be swallowed by the patient or to be inserted into the body cavity with a laparoscope or an endoscope. Once inside the body, the device can be attached to the wall of the vessel or cavity by using the securing element. The device provides localized single point illumination, visualization, or sensory monitoring where needed by the physician. The device can also have multiple light sources, viewing elements and/or sensors attached to a single capsule or several devices could be used to illuminate and/or visualize or monitor multiple points along the inner surface of a body cavity.

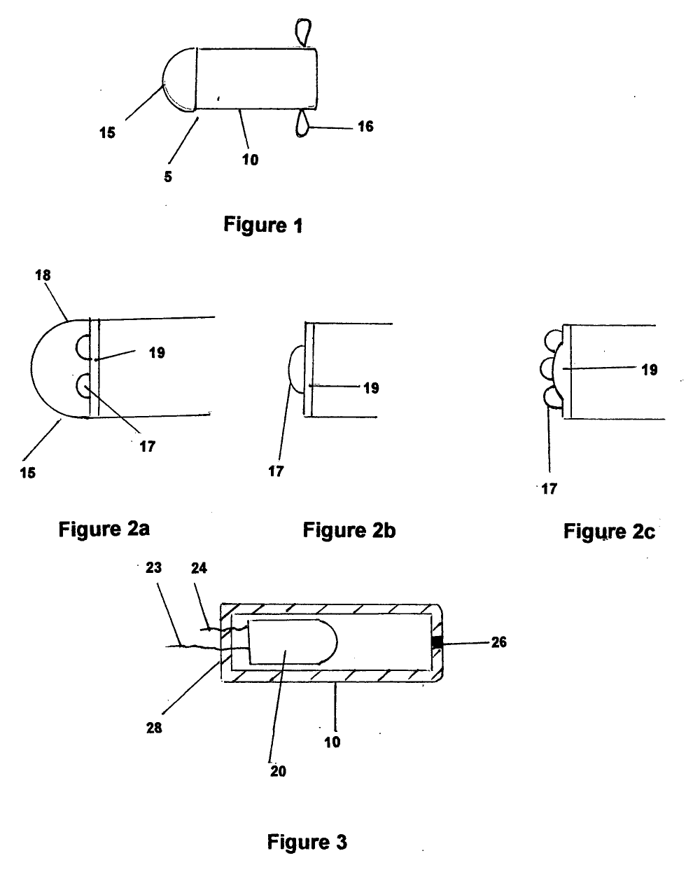

FIGS. 1-19 depict embodiments of self contained optical and sensory elements suitable for placement into a body cavity by swallowing or with an endoscope. The depicted apparatus (as well as the other embodiments depicted and/or described herein) may be used to provide illumination and/or monitoring during an endoscopic surgical procedure.

In all figures, even though an optical element is described, it should be equally understood that a sensor or sensory element or multiple sensors could be utilized to monitor a condition or multiple conditions within a hollow cavity of the body. By way of discussion, monitoring a hollow cavity of a body may include optical monitoring such as with a camera that provides direct visualization of the optical state or condition of a hollow body. Likewise monitoring can be interpreted to mean chemical, physical or audio monitoring of a hollow cavity through the use of various types of sensors that monitor and report a physical state or condition of a hollow cavity.

FIG. 1 illustrates the device 5 which includes a housing 10 with an optical element 15 coupled to it. The housing 10 is shown with a generally elliptical or tubular shape but any shape and size convenient for placement into the body cavity is satisfactory. The device 5 may be placed into the body cavity of interest by an endoscope through the working channel of that endoscope. Alternatively the device 5 may be placed by using an instrument to grasp the device such as forceps, loops, or baskets that are first positioned into and exit from the working channel of an endoscope. These grasping device can be used to hold the device 5 in a position just distal to the tip of the endoscope and can release the device 5 when access to the body cavity has been achieved. Alternatively the device 5 may be swallowed by a patient. In this method the device is swallowed and later retrieved and positioned by an endoscope. In the case of a device that is to be swallowed, a generally round shape may be more conducive to swallowing by the patient. Alternatively a cylinder with rounded ends similar to a pill capsule may also be used. The housing 10 may be coated with substances that facilitate the passage of the device into the body cavity. Examples of such coatings are gels, hydrophilic coatings, or other lubricious coatings that reduce the coefficient of friction.

The optical element 15 is shown at one end of the housing 10 but is anticipated that the illumination or optical sensor element 15 could be located anywhere along the body of the housing 10. Also multiple illumination or optical elements 15 could also be located on a single housing 10. In another embodiment, the housing 10 has multiple attachment ports where a single illumination or optical element 15 or more than one optical element 15 can be plugged in and electrical connections made. This embodiment permits the housing 10 to be delivered first and then the optical element 15 to be attached later at the most optimum location on the housing. By way of example, if the optical element includes a light source, additional light sources could be attached if more illumination was needed.

The housing has a securement element 16 that may be used for securing the device to the cavity wall. The device 5 is shown in a cylindrical shape with the securement elements 16 at one end and the optical element 15 at the other but the securement elements 16 could be attached to the device 5 at any convenient location. The housing 10 may be made of biocompatible materials such as metal or plastic.

The optical element 15 includes a light source 17 as shown in FIGS. 2a-c, which may be used to illuminate at least a portion of the body cavity. The light source 17 is intended to provide general illumination of the body cavity by providing white light that is scattered to cover a wide area. However a more focused beam of light is possible and the use of single wavelengths or multiple wavelengths of light are also anticipated. As shown in FIG. 2a, the optical element 15 includes a light source 17 that is located inside a dome housing 18. The light source 17 may be a light bulb that uses a filament that emits light as electrical current is passed through the filament. The light source 17 may also be an LED or light emitting diode. LED's are particularly useful because the LED emits a high intensity light source and is small so that it is suitable for use with as a compact light source 17. The optical element 15 may use one or more light sources 17 placed inside of the dome housing 18. This dome housing 18 can modify the intensity or scattering of the light emitted by the light source 17. For example, the dome housing 18 could be designed to focus the light from the light source 17 into a single concentrated beam or could also be designed to scatter light over a broad area if desired. In one embodiment, the light source 17 and the dome housing 18 may focus light in a first direction and be attached to a plate 19 in such a way that the dome housing 18 can swivel about the plate 19. The dome housing 18 can then be swiveled about the plate 19 to focus the light in a second direction without moving the entire device. By rotating the dome housing 18, light can be directed where needed. The dome housing 18 shown can be sealed to the plate 19 to prevent liquids from entering the inner space of the lens 18 to protect the light source 17 from exposure to body fluids. The lens 18 may be formed from materials such as glass or plastic.

However, as shown in FIGS. 2b and 2c, one or more light sources 17 can be mounted to the plate 19 without a dome housing 18. This could potentially reduce the device's complexity, size and cost. In this embodiment the light sources and their associated electrical connections would be sealed so that they were capable of direct exposure to the internal environment. The light sources 17 could be mounted in various positions onto the plate 19 as shown in FIG. 2c so as to provide illumination and viewing in multiple directions simultaneously or to provide general illumination to a wide area.

As shown in FIG. 3, a power source 20 provides the electricity to activate the light source 12. The power source 20 is a battery sealed inside the housing 10. The power source 20 is electrically connected to the light source 17 using insulated wires 23 and 24. In one embodiment the housing 10 includes a switch 26 to turn on the apparatus before or after placement into the body.

The power source 20 and the light source 17 are joined together as shown in FIG. 1 with the plate 19 attached to one end 28 of the power source 20 and providing electrical connections 23 and 24 in between. The plate 19 and the housing 10 are permanently or detachably joined together with a union that prevents body fluids from contacting the battery or destroying the integrity of the electrical connections.

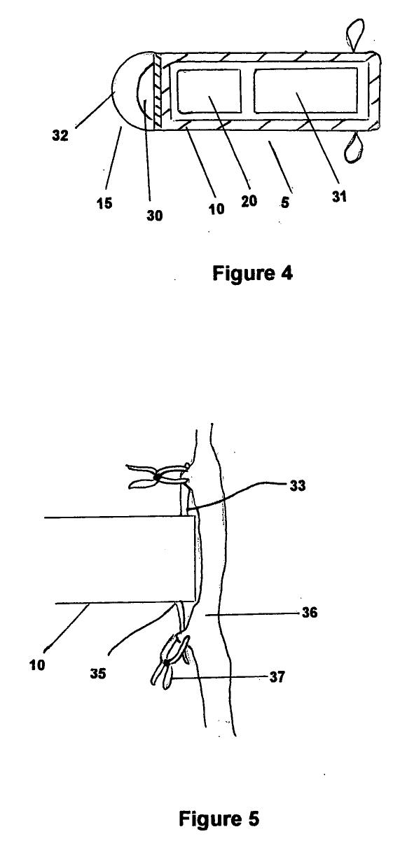

In another embodiment, the optical element 15 as shown in FIG. 4 includes a viewing element 30 which can be used to view a portion of the body cavity. The viewing element 30 is designed to provide an operator an image of the body cavity so that surgical interventions or examinations may be completed without the need for the use of an endoscope. In a preferred embodiment, the viewing element 30 may be used to observe a particular anatomical structure over a length of time. This sort of observation would be difficult with an endoscope placed down a patient's esophagus. With a remote viewing element located in the body cavity, the viewing element 30 may be secured to a portion of the body cavity so that it is focused on a particular anatomical structure or other area of interest. The viewing element 30 may be a camera or an electronic imaging sensor. Examples of electronic imaging sensors are CCD or CMOS (complementary metal oxide semiconductor) chips. The viewing element 30 also includes a transmitter 31 that is required to transmit the images captured by the viewing element 30 to a receiver (not shown) located outside the patient's body. The viewing element 30 views the image, converts the image to an electronic signal and then the transmitter 31 sends these electronic signals to the receiver. The optical element 15 may include a lens 32 that modifies or magnifies the image for the viewing element 30. The lens 32 could be a wide angle lens to permit wide angle viewing of the body cavity. Alternately the lens 32 could use focusing optics to narrow the angle of view or magnify the image. The lens may be changeable so that different lenses 32 could be used for different needs.

In a preferred embodiment, the optical element 15 includes both a light source 17 and a viewing element 30. In this configuration the optical element 15 would be capable of illuminating the body cavity, capturing images of the body cavity and transmitting the images to a receiver located outside the patient.

It is important to provide a method of securing the light source and/or optical sensor to the inside of the body where it is intended to be used. This feature allows the physician to attach and detach the optical element 15 as needed, to focus the light source 17 and/or viewing element 30 where needed and to provide a stable platform despite moving body fluids, vessel walls or organs. The securement elements 16 should be convenient, sturdy and add minimal bulk to the profile of the light source. Several securement elements 16 are depicted in FIGS. 5-10, however it will be understood by those skilled in the art that the invention extends past the attachment embodiments specifically presented to other alternative embodiments and/uses of the invention thereof.

FIG. 5 depicts one or more loops 33 attached to one end 34 of the housing 10. The loops 33 can be made of natural or synthetic materials such as wire, plastic, string, shape elastic metal or suture material. They can be made of materials that dissolve or reabsorb over time so that the device 5 could detach and be sloughed off in the case of usage in the bowel. They can be attached to the housing 10 at various locations as desired. For example, the attachment point 35 could be placed at different positions to permit the possibility of attaching loops as the physician required. In a preferred embodiment the loops would be attached before placement into the body. In another embodiment, loops or other attachment embodiments could be attached after placement into the body. The loops 33 could be attached to a wall 36 of the body cavity, vessel or organ with endoscopic clips 37. These clips would pinch the tissue and the loop 33 together to securely tether the loop 33 to the tissue. The use of multiple tether points as shown would provide a secure fixation of the light source. These clips 37 could be unfastened to facilitate removal or repositioning of the light source if required.

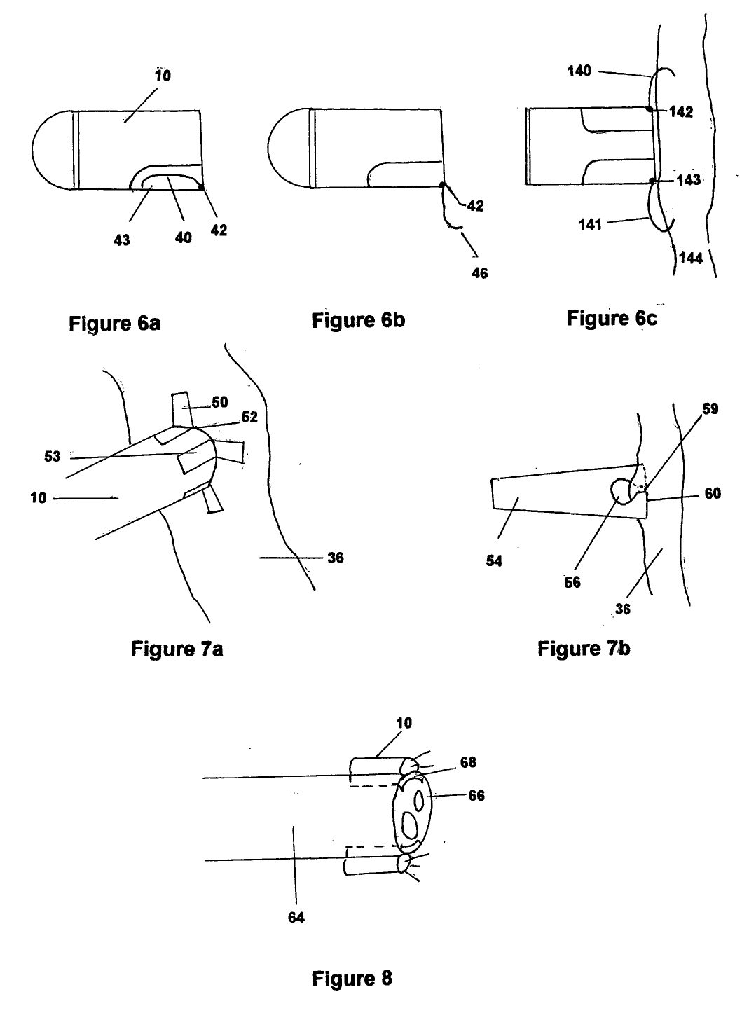

FIGS. 6a through 6c describe an another embodiment of a securement element 16 utilizing one or more hooks, barbs, or pins 40 that are attached to the housing 10 at pivot point 42. The housing 10 side could be recessed 43 at the location of the hook 40 shown in FIG. 6a to provide a uniform outside diameter of the housing 10. This would facilitate the smooth passage down the working lumen of an endoscope, for example. The hook 40 is in a first or closed position as shown in FIG. 6a for placement into the body or into the working lumen of the endoscope. As the device 5 is deployed, the hook 40 moves to a second or open position, FIG. 6b. In this position, the sharp end 46 can imbed itself into tissue. FIG. 6c describes another embodiment of an attachment apparatus utilizing two or more hooks or barbs 140 and 141 that are attached to the housing 10 at pivot points 142 and 143. The hooks are in a first or closed position (not shown) for placement into the body or into the working lumen of an endoscope. As the device 5 is deployed, the hooks move to a second or open position, FIG. 6c. In this position, the sharp ends travel past one another to pinch tissue 144 in between the two ends. The hooks can be spring loaded or manually deployed. The attachment of the hooks, barbs, or pins can be facilitated by the use of suction or magnets. Suction can be generated through the endoscope or a catheter attachment to the device.

FIGS. 7a and 7b show another embodiment of a securement element 16 utilizing one or more tabs 50 that unfold from the side of the housing 10 at one or more pivot points 52. The tabs 50 are in a first or closed position (not shown) for delivery of the device to a body cavity or vessel and then unfold to a second position shown in FIG. 7a along the wall 36 of the body cavity for deployment. In a first position, the tabs 50 are positioned in a recessed cavity 53 located at one end of the housing 10. In this position the tabs 50 are flush with the exterior diameter of the housing 10 and can pass through the working lumen of an endoscope. As the device 5 is deployed, the tabs 50 move to a second or open position. The tabs 50 provide an alternate method to fasten the device 5 to the wall of a body cavity, vessel or organ with endoscopic clips. The tabs 50 can be made from plastic, cloth, or metal and can be placed at multiple locations on the container. The tabs 50 can be made of materials that dissolve or reabsorb over time so that the tiny light sources can detach and be sloughed off in the case of usage in the bowel. FIG. 7b depicts an alternative embodiment of a securement element. In this embodiment tab 54 is shown with a hole 56 that is connected with the outside edge of the tab by a slot 59. This slot can be spread apart so that the ends 60 are separated from each other by a small distance. If tissue 36 is brought between the displaced tab ends 60 and the tab ends are released the tissue will be pinched in-between the tab ends 60.

In another embodiment the device 5 can be secured to the wall of a body cavity using adhesive. The adhesive secures the device 5 to the wall until the cells on the surface regenerate at which time the device 5 would be sloughed off as described previously. The adhesive may have chemical properties that enable activation in the body cavity.

In another embodiment illustrated in FIG. 8, the housing 10 can be temporarily attached to the end of the endoscope 64 using magnets after the endoscope is placed into the body cavity. As shown in FIG. 8, one or more devices 5 can be attached to the distal end 66 of an endoscope 64. The end 66 of the endoscope may contain magnets 68 and the housing 10 be made of metal. When the housing 10 gets near the magnetic end of the endoscope 66 the housing 10 is drawn to the endoscope 64 and is attached through magnetic attraction.

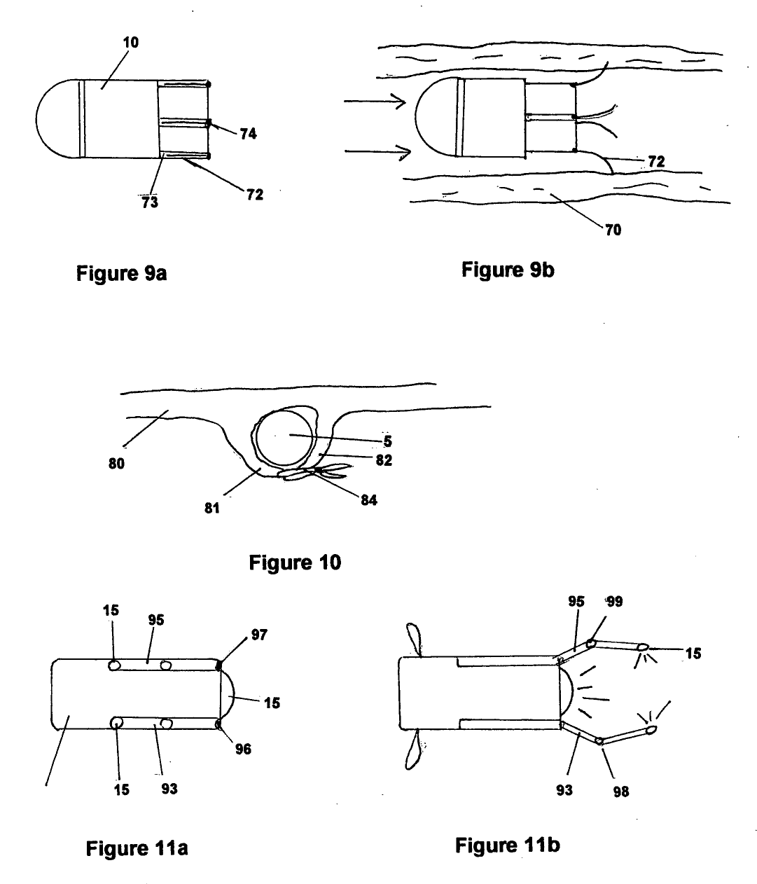

In another embodiment, the device 5 can be deployed into vessels 70 of the body utilizing one or more arms 72 as shown in FIGS. 9a and 9b. The arms 72 are in a first or closed position as shown in FIG. 9a for placement through the working lumen of the endoscope. In a first position, the arms 72 are positioned in a recessed cavity 73 located at one end of the housing 10. In this position the arms 72 are flush with the exterior diameter of the housing 10 and can pass through the working lumen of an endoscope. As the device 5 is deployed, the arms rotate about pivot points 74 and move to a second or open position as shown in FIG. 9b. In this position the arms wedge into the sides of a vessel 70 and anchor themselves against any movement within the vessel 70. The device 5 could be placed in a downstream position as shown, from an endoscope to illuminate an upstream area. Alternatively the device could be positioned upstream from the endoscope to illuminate a downstream area with the endoscope positioned between the arms 72. In this orientation the arms 72 can deploy in a direction opposite to that shown in FIG. 9b. Alternatively, if the optical element includes a viewing element, the device 5 can be used to illuminate and/or visualize the vessel structures as necessary. The arms can be spring loaded or manually deployed as needed.

It is also possible to secure the device 5 into a cavity, organ or tubular structure (such as any part of the tubular gastrointestinal tract, a duct, or vessel) by using tissue 80 as shown in FIG. 10. In this figure, the device 5 is shown as viewed from the end. If the device 5 has a cylindrical shape, tissue ends 81 and 82 can be grasped by the end of a grasping instrument that is placed down the working lumen of the endoscope. The tissue can be folded onto itself and clipped using an endoscopic clip 84. This embodiment secures the device 5 without the need for any additional loops, tabs or hooks.

FIGS. 11a and 11b depict an alternative embodiment of the device 5. It is often advantageous to be able to view a portion of a body cavity with more than one angle or view. Sometimes directing a light source at a different optical axis than the viewing element may facilitate a better image. The embodiment depicted consists of more than one optical element 15 attached to a single housing 10. Multiple optical elements 15 in various combinations including light sources and/or viewing elements are attached to arms 93 and 95. The arms are attached to pivot points 97 and 98 that permit the arms to fold down into a recessed cavity (not shown) so that they are flush with the outside of the housing 10 in the initial or closed position as shown in FIG. 11a. In this position the device is suitable for placement down the working channel of an endoscope or by swallowing. The arms rotate about pivot points 97 and 98 which are positioned near a primary optical element 15. This configuration, FIG. 11b, illustrates how this design would provide more illumination and/or visualization over a broader field than a single light source and/or viewing element. In this embodiment, the arms 93 and 95 deploy manually by manipulation with endoscopic instruments. As shown the arms may have additional articulation or pivot points 98 and 98. These articulation points permit greater freedom to position the optical elements as required. In another embodiment, the arms self-deploy when the light source and/or viewing element exits the distal end of the endoscope. In still another embodiment, the arms deploy after removal of a retention sleeve (not shown).

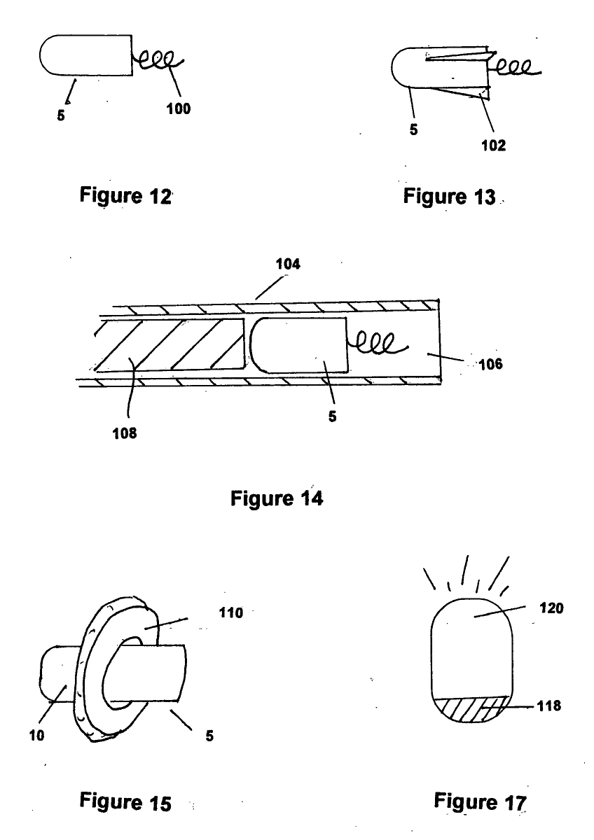

In another embodiment, FIG. 12, the device 5 can be secured to the wall of a body cavity using a helicoil or screw 100. The helicoil or screw 100 is coupled to the housing at one end and can attach the device 5 to a wall of a body cavity by twisting it into the wall. The screw 100 can be attached so that the attachment and unattachment is performed by an operator. Alternatively, the screw 100 could be made from bioabsorbable materials so that the screw 100 dissolves or is reabsorbed by the body after a period of time. When this occurs, the screw 100 disappears and the device 5 is sloughed off

In another embodiment, the stability of the device 5 against the wall can be optimized with legs 102 that project from the housing 10 of the device 5 in a radial orientation. In a first position, the legs 102 are positioned in a recessed cavity in the housing and a screw 100 is coupled to one end of the housing 10. In this position the legs 102 are flush with the exterior diameter of the housing 10 and can pass through the working lumen of an endoscope. The legs 102 move to a second or open position as shown in FIG. 13 by a spring loaded release mechanism or manual deployment. In the open position the legs 102 stabilize the device 5 against the wall of the body cavity in an orientation that is generally perpendicular to the wall of the body cavity.

FIG. 14 depicts a sheath or catheter 104 for insertion of device 5 through the working channel of an endoscope and deployment in a body cavity. In a first position as shown the device 5 is fully retracted into the inner lumen 106 of the catheter 104 for insertion through the working channel of the endoscope. The catheter 104 has a pusher tube 108 located inside. The pusher tube 108 is utilized to push the device 5 out of the distal end of the inner lumen 106 of the catheter 104. The device 5 may alternatively be deployed by retracting the catheter 104 while keeping the device 5 stationary with the use of the pusher tube 108. The catheter 104 may also serves as a restraining element to hold a spring-loaded component such as legs shown in FIG. 13 or barbs in a closed position. The catheter 104 may be designed so that it can transmit torque along its length. For example, a clockwise torque applied at the proximal end of the catheter outside the body would cause the distal end to rotate in a clockwise manner. This ability to transmit torque may be useful to screw the helical screw into the walls of the body cavity.

In another embodiment shown in FIG. 15 an inflatable balloon 110 can be coupled to the housing 10 of the device 5. The balloon 110 has a valve that permits inflation and deflation of the balloon 110 with a fluid. This balloon 110 can be inflated when the device is used in a small vessel or lumen. The inflated balloon 110 would possibly secure and center the device 5 to the vessel wall. Upon completion of the procedure the balloon 110 could be deflated to using the valve or the balloon could be torn or cut with graspers to release the fluid and deflate the balloon.

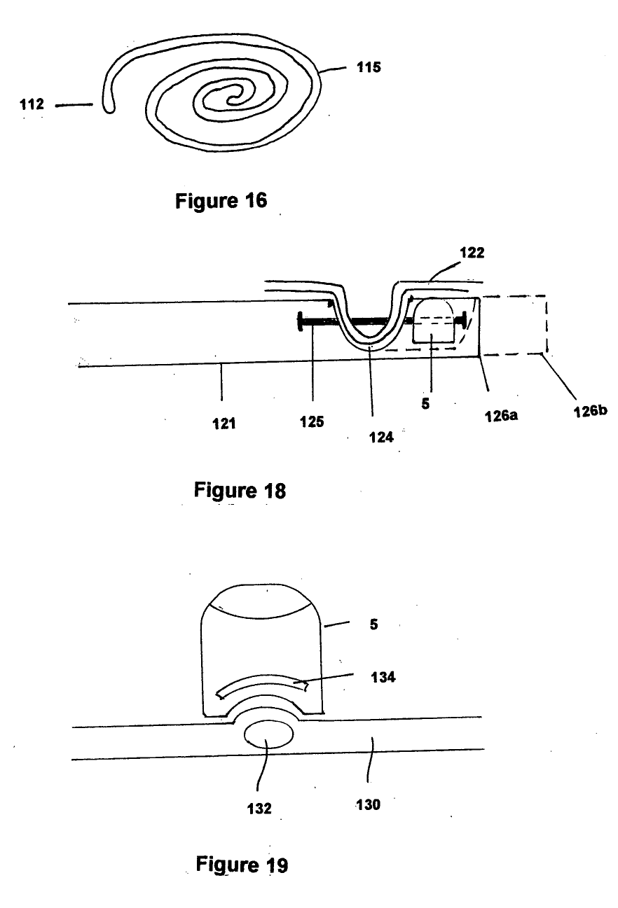

In another embodiment shown in FIG. 16 the device 112 is comprised of a shaped tube 115. The tube 115 can be delivered through the working lumen of an endoscope. The tube 115 is straightened out and passed through an endoscope. Once inside the body cavity, the tube 115 self coils to a pre-formed coil configuration. The coil configuration helps prevent the device 112 from moving out of a body cavity. If the device is placed in the stomach, the coiled configuration helps prevent the device 112 from passing through the Pylorus and into the small intestine. The tube can contain chemicals that react and illuminate the body cavity. Various chemicals give off light when brought into contact with one another. This light may be used to illuminate otherwise dark body cavities. The tube may have multiple chambers containing a chemical A and a chemical B. When these chambers are ruptured, the two chemicals combine giving off light. Some examples of these types of chemicals are peroxide and a phenyl ester or luciferin and ATP. These examples are not meant to be limiting and any chemicals that emit light when combined would be sufficient. The tube may also be coated with one chemical and rely on an interaction with a second material found in the body to cause the chemical reaction necessary to emit light. The tube itself as well as the contents of the tube may be biocompatible. The tube may be made of bioresorbable material. A non-resorbable tube may be used to house non-biocompatible components of an illuminating or sensor device or energy source such as a battery.

In another embodiment shown in FIG. 17 the device 5 has a weighted portion 118 that is denser and heavier than the other parts of the device 5. The heavier portion is attracted by gravity more than the rest of the device 5 so the heavy portion 118 is generally aligned toward the ground. This aligns the optical element or sensor located at the opposite end 120 of the device in a direction generally perpendicular to the wall of the body cavity. This can be particularly useful when the patient is lying on a procedure table and much of the body cavity of the body has sides aligned parallel to the ground.

In the embodiment shown in FIG. 18, a delivery device 121 is attached to the end of an endoscope. Suction is used to draw a portion of the wall of a body cavity 122 into a hollow chamber 124 of the delivery device 121. Once tissue is drawn inside the hollow cavity 124, an attachment between the body cavity wall 122 and the device 5 is accomplished by placing a T-Tag, a staple or a pin 125 through a soft portion of the device 5 and into the tissue pulled into the hollow chamber 124. In this position the device 5 and the tissue walls are securely attached. Once attached, the end of the delivery device is extended from a first position 126a to a second position 126b. When the end of the delivery device is extended as shown, the length of the hollow chamber 124 is also increased. This enlarged hollow chamber is larger than the tissue and the device 5 so when suction is stopped, the tissue with the attached device is released from the delivery device 121.

In another embodiment, the housing 10 may be secured to the body cavity wall using magnetic attraction. As shown in FIG. 19, a magnet or magnetic material 132 is first placed into the site on the body cavity wall 130 that is the intended site for placement of an optical element or sensor. This magnet or magnetic material 132 might be injected into the sub mucosa of the soft tissue wall 130. The device 5 has an oppositely charged magnet 134 positioned inside the housing 10. When the housing 10 of the device 5 is comes into close proximity to the imbedded magnet or magnetic material 132 which has been previously implanted in the soft tissue wall, the magnets which have opposite polarity are attracted to each other and the device 5 is magnetically coupled to the body cavity wall 130.

This invention has been described and specific examples of the invention have been portrayed. The use of those specifics is not intended to limit the invention in anyway. Additionally, to the extent that there are variations of the invention, which are within the spirit of the disclosure or equivalent to the inventions found in the claims, it is my intent that this patent will cover those variations as well.

1. An apparatus to facilitate viewing an interior body surface of a patient, said apparatus comprising:
a housing with an optical element coupled to the housing;
a battery located in said housing to provide power to the optical element;
and a securement element to position the apparatus within the body cavity.

2. The apparatus of claim 1 wherein the housing and optical element can be placed through a working lumen of an endoscope.

3. The apparatus of claim 1 where the optical element includes at least one light source.

4. The apparatus of claim 3 wherein the at least one light source has a filament.

5. The apparatus of claim 3 wherein the at least one light source is an LED.

6. The light source of claim 3 wherein the at least one light source generates light using a chemical reaction.

7. The light generating chemical reaction of claim 6 whereby the reaction includes peroxide and a phenyl ester.

8. The light generating chemical reaction of claim 6 whereby the reaction contains a luciferin and ATP.

9. The apparatus of claim 3 wherein the at least one light source includes a lens to modify the diffraction of light from the light source.

10. The light source of claim 3 wherein the at least one light source can be articulated relative to said housing.

11. The securement element described in claim 1 wherein said securement element includes a barbed hook, a loop or a tab.

12. The tab of claim 11 wherein said tab is split so that it can pinch said interior body surface.

13. The securement element described in claim 1 wherein said securement element includes a cord which extends to the exterior of a patient's body.

14. The securement element described in claim 1 wherein said securement element includes a helicoil, shaped tube, screw, magnet, balloon, clip or a T-Tag.

15. The securement element described in claim 1 wherein said securement element includes a suction chamber.

16. The optical element of claim 1 wherein the optical element includes at least one viewing element and a transmitter so that images of the interior surface of the body can be transmitted to a receiver located outside of the patient's body.

17. The viewing element of claim 16 wherein the at least one viewing element can be articulated relative to said housing.

18. The viewing element of claim 16 wherein the viewing element includes a camera.

19. The viewing element of claim 16 wherein the viewing element includes lens and a CCD.

20. An apparatus for illuminating an interior body surface of a patient, said apparatus comprising:
a housing, said housing sized to be placed into proximity to the interior body surface;
a light source;
a battery located in said housing to provide power to the light source; and
a securement element to position the apparatus within the body cavity.

21. A self contained device for remotely viewing a body cavity, said device comprising:
a housing, said housing sized to be placed into said body cavity;
an optical element coupled to the housing;
a power source to provide power to the optical element; and
a securement element to position the apparatus within the body cavity.

22. An apparatus to remotely measure a condition of a body cavity of a patient, said apparatus comprising:
a housing with a sensor element coupled to the housing;
a battery located in said housing to provide power to the sensor element; and
a securement element to position the apparatus within the body cavity.

23. The apparatus of claim 22 whereby the sensor measures a Ph or a temperature of the body cavity.

24. The apparatus of claim 22 whereby the sensor measures a force of contractility or an electrolyte or a protein concentration of the body cavity.

25. A method of viewing a body cavity of a patient comprising the steps of:
placing a housing inside the body cavity, said housing containing a power source, a viewing element and a transmitter;
securing the housing to the body cavity;
using said viewing element to view the body cavity; and
transmitting the images to a receiver outside the patient.

26. The method of claim 25 whereby the housing contains a light source and the step includes illuminating the body cavity with said light source.

27. A method of illuminating a body cavity of a patient comprising the steps of:
placing a housing inside the body cavity, said housing containing a power source and a light source;
securing the housing to the body cavity; and
activating the light source to provide light inside the body cavity.

28. A method of measuring a condition of a body cavity of a patient comprising the steps of:
placing a housing inside the body cavity, said housing containing a power source, at least one sensor element and a transmitter;
securing the housing to the body cavity;
measuring said condition of a body cavity; and
transmitting the information to a receiver outside the patient.
